(19) United States
(12) Patent Application Publication
(10) Pub. No.: US 2011/0230893 A1
(43) Pub. Date: Sep. 22, 2011

(54) SYSTEMS AND METHODS FOR MAKING AND USING ELECTRICAL STIMULATION SYSTEMS HAVING MULTI-LEAD-ELEMENT LEAD BODIES

(75) Inventor: John Michael Barker, Ventura, CA (US)

(73) Assignee: Boston Scientific Neuromodulation Corporation, Valencia, CA (US)

(21) Appl. No.: 13/044,917

(22) Filed: Mar. 10, 2011

Related U.S. Application Data
(60) Provisional application No. 61/315,861, filed on Mar. 19, 2010.

(51) Int. Cl. A61N 1/36; A61N 1/04; A61B 19/00

(52) U.S. Cl. 606/129; 607/148; 607/2

(57) ABSTRACT

A lead for providing electrical stimulation of patient tissue includes a distal lead element and three proximal lead elements. The distal lead element includes at least twenty electrodes and defines a stylet lumen and a plurality of conductive wire lumens having triangular transverse cross-sectional shapes. Each of the three proximal lead elements includes a plurality of terminals and defines a plurality of conductive wire lumens. Each of the conductive wire lumens has a round transverse cross-sectional shape. A junction couples the distal lead element to the proximal lead elements. Conductive wires couple each of the electrodes of the distal lead element to at least one of the terminals of at least one of the proximal lead elements. The conductive wire lumens disposed on the distal lead element are configured and arranged to receive a plurality of the conductive wires.

### CROSS-REFERENCE TO RELATED APPLICATIONS

This application claims the benefit under 35 U.S.C. §119(e) of U.S. Provisional Patent Application Ser. No. 61/315,861 filed on Mar. 19, 2010, which is incorporated herein by reference.

### FIELD

The present invention is directed to the area of implantable electrical stimulation systems and methods of making and using the systems. The present invention is also directed to implantable electrical stimulation leads having lead bodies with multiple lead elements, as well as methods of making and using the leads, lead bodies, lead elements, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a lead for providing electrical stimulation of patient tissue includes a distal lead element having a longitudinal axis and three proximal lead elements. The distal lead element includes at least twenty electrodes disposed on the distal lead element, a stylet lumen extending along at least a portion of the longitudinal axis of the distal lead element, and a plurality of conductive wire lumens extending along at least a portion of the longitudinal axis of the distal lead element. Each of the conductive wire lumens has a triangular transverse cross-sectional shape. Each of the three proximal lead elements have a longitudinal axis and include a plurality of terminals disposed on the proximal lead element, and a plurality of conductive wire lumens extending along at least a portion of the longitudinal axis of the proximal lead element. Each of the conductive wire lumens has a round transverse cross-sectional shape. A junction couples the distal lead element to the proximal lead elements. A plurality of conductive wires couple each of the plurality of electrodes disposed on the distal lead element to at least one of the plurality of terminals disposed on at least one of the proximal lead elements. The plurality of conductive wire lumens disposed on the distal lead element are configured and arranged to receive a plurality of the conductive wires.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2A is a schematic view of one embodiment of a proximal portion of a lead and a control module of an electrical stimulation system, according to the invention;

FIG. 2B is a schematic view of one embodiment of a proximal portion of a lead and a lead extension of an electrical stimulation system, according to the invention;

FIG. 3 is a schematic perspective view of one embodiment of a lead having a distal lead element coupled to proximal lead elements by a junction, according to the invention;

FIG. 4 is a schematic transverse cross-sectional view of one embodiment of the distal lead element of FIG. 3, according to the invention;

FIG. 5 is a schematic transverse cross-sectional view of one embodiment of one of one of the proximal lead elements of FIG. 3, according to the invention;

FIG. 6 is a schematic perspective exploded view of one embodiment of a lead introducer configured and arranged to facilitate implantation of an electrical stimulation system into a patient, according to the invention;

FIG. 7 is a schematic perspective view of one embodiment of the lead introducer of FIG. 6, according to the invention;

FIG. 8 is a schematic perspective close-up view of one embodiment of a distal end of the lead introducer of FIG. 6, according to the invention;

FIG. 9 is a schematic perspective longitudinal cross-sectional view of one embodiment of a proximal end of the lead introducer of FIG. 6, according to the invention;

FIG. 10A is a schematic perspective view of one embodiment of a lead and an outer insertion needle of the lead introducer of FIG. 6, the outer insertion needle defining an open channel extending along a length of the outer insertion needle, the open channel configured and arranged to receive the lead, according to the invention;

FIG. 10B is a schematic transverse cross-sectional view of several exemplary embodiments of the open channel of the outer insertion needle of FIG. 10A, according to the invention.

FIG. 11 is a schematic perspective view of one embodiment of an obturator removed from the lead introducer of FIG. 6, according to the invention;

FIG. 12 is a schematic perspective view of one embodiment of an inner insertion needle removed from the lead introducer of FIG. 6 and replaced with the lead of FIG. 3, according to the invention;

FIG. 13 is a schematic perspective view of one embodiment of a splitable member of the lead introducer of FIG. 6 being split apart to remove the splitable member from the lead of FIG. 3, according to the invention;

FIG. 14A is a schematic longitudinal cross-sectional view of one embodiment of a lead introducer with a removable retaining member disposed over a split-release insertion needle, according to the invention;

FIG. 14B is a schematic transverse cross-sectional view of one embodiment of the lead introducer of FIG. 14A, according to the invention; and

FIG. 15 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to implantable electrical stimulation leads having lead bodies with multiple lead elements, as well as methods of making and using the leads, lead bodies, lead elements, and electrical stimulation systems.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; and 6,741,892; and U.S. Patent Applications Publication Nos. 2003/0114905, 2005/0165465, 2007/0150036; 2007/0161294; 2007/0219595; 2007/0239243; 2007/0150007; and 2008/0071320, and U.S. patent application Ser. No. 11/238,240, all of which are incorporated by reference.

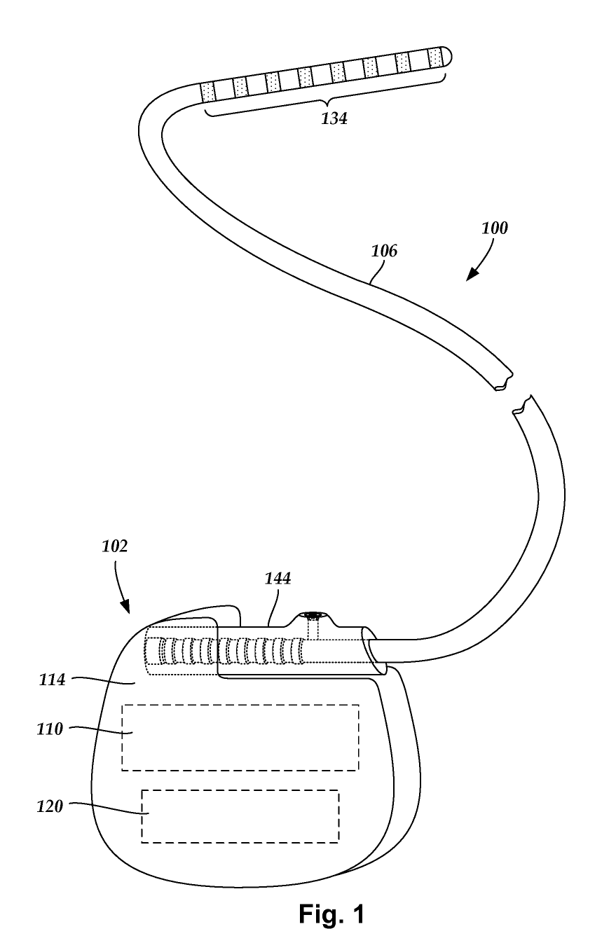

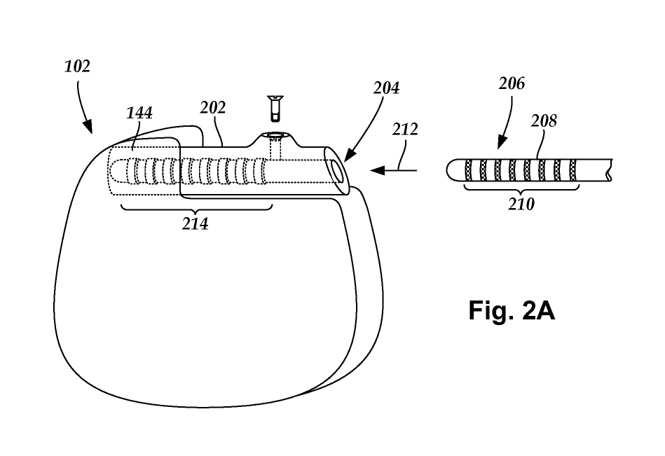

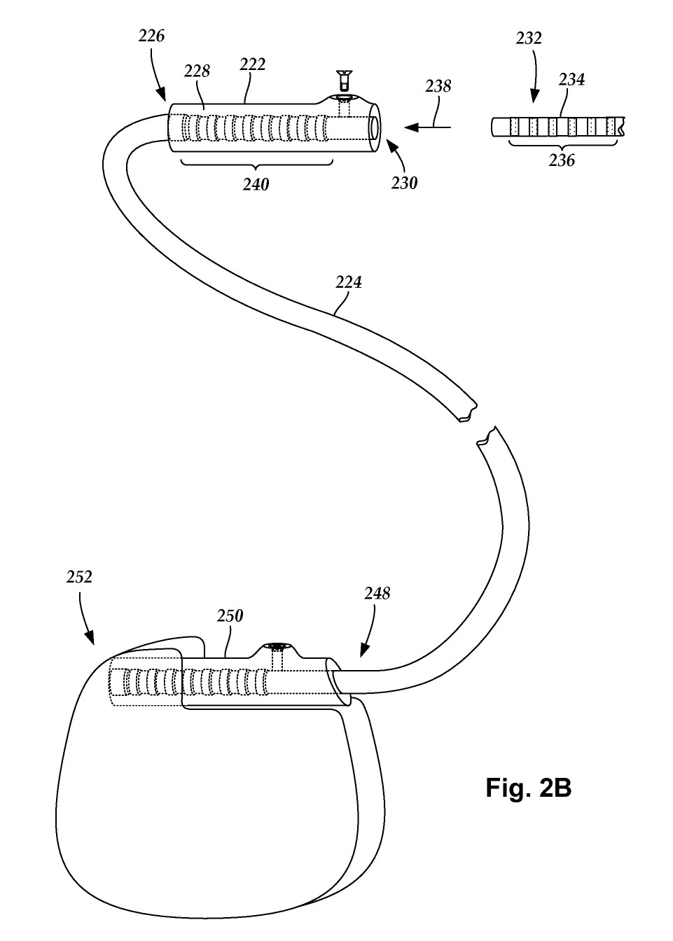

FIG. 1 illustrates schematically one embodiment of an electrical stimulation system 100. The electrical stimulation system includes a control module (e.g., a stimulator or pulse generator) 102 and at least one lead 106 coupled to the control module 102. Each lead 106 typically includes an array of electrodes 134. The control module 102 typically includes an electronic subassembly 110 and an optional power source 120 disposed in a sealed housing 114. The control module 102 typically includes a connector 144 (FIG. 2A, see also 222 and 250 of FIG. 2B) into which the proximal end of the one or more leads 106 can be plugged to make an electrical connection via conductive contacts on the control module 102 and terminals (e.g., 210 in FIGS. 2A and 236 of FIG. 2B) on each of the one or more leads 106. In at least some embodiments, a lead is isodiametric along a longitudinal length of the lead 106. In addition, one or more lead extensions 224 (see FIG. 2B) can be disposed between the one or more leads 106 and the control module 102 to extend the distance between the one or more leads 106 and the control module 102 of the embodiment shown in FIG. 1.

The electrical stimulation system or components of the electrical stimulation system, including one or more of the leads 106 and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. The number of electrodes 134 in the array of electrodes 134 may vary. For example, there can be two, four, six, eight, ten, twelve, fourteen, sixteen, or more electrodes 134. As will be recognized, other numbers of electrodes 134 may also be used.

The electrodes of one or more leads 106 are typically disposed in, or separated by, a non-conductive, biocompatible material such as, for example, silicone, polyurethane, polyetheretherketone (“PEEK”), epoxy, and the like or combinations thereof. The leads 106 may be formed in the desired shape by any process including, for example, molding (including injection molding), casting, and the like. The non-conductive material typically extends from the distal end of the one or more leads 106 to the proximal end of each of the one or more leads 106.

Terminals (e.g., 210 in FIGS. 2A and 236 of FIG. 2B) are typically disposed at the proximal end of the one or more leads 106 of the electrical stimulation system 100 for connection to corresponding conductive contacts (e.g., 214 in FIGS. 2A and 240 of FIG. 2B) in connectors (e.g., 144 in FIGS. 1-2A and 222 and 250 of FIG. 2B) disposed on, for example, the control module 102 (or to conductive contacts on a lead extension, an operating room cable, or an adaptor). Conductor wires (not shown) extend from the terminals (e.g., 210 in FIGS. 2A and 236 of FIG. 2B) to the electrodes 134. Typically, one or more electrodes 134 are electrically coupled to a terminal (e.g., 210 in FIGS. 2A and 236 of FIG. 2B). In at least some embodiments, each terminal (e.g., 210 in FIGS. 2A and 236 of FIG. 2B) is only connected to one electrode 134. The conductor wires may be embedded in the non-conductive material of the lead 106 or can be disposed in one or more lumens (not shown) extending along the lead 106. In some embodiments, there is an individual lumen for each conductor wire. In other embodiments, two or more conductor wires may extend through a lumen. There may also be one or more lumens (not shown) that open at, or near, the proximal end of the lead 106, for example, for inserting a stylet rod to facilitate placement of the lead 106 within a body of a patient. Additionally, there may also be one or more lumens (not shown) that open at, or near, the distal end of the lead 106, for example, for infusion of drugs or medication into the site of implantation of the one or more leads 106. In at least one embodiment, the one or more lumens may be flushed continually, or on a regular basis, with saline, epidural fluid, or the like. In at least some embodiments, the one or more lumens can be permanently or removably sealable at the distal end.

In at least some embodiments, leads are coupled to connectors disposed on control modules. In FIG. 2A, a lead 208 is shown configured and arranged for insertion to the control module 102. The connector 144 includes a connector housing 202. The connector housing 202 defines at least one port 204 into which a proximal end 206 of a lead 208 with terminals 210 can be inserted, as shown by directional arrow 212. The connector housing 202 also includes a plurality of conductive contacts 214 for each port 204. When the lead 208 is inserted into the port 204, the conductive contacts 214 can be aligned with the terminals 210 on the lead 208 to electrically couple the control module 102 to the electrodes (134 of FIG. 1) disposed at a distal end of the lead 208. Examples of connectors in control modules are found in, for example, U.S. Pat. No. 7,244,150 and U.S. patent application Ser. No. 11/532,844, which are incorporated by reference.

In FIG. 2B, a connector 222 is disposed on a lead extension 224. The connector 222 is shown disposed at a distal end 226 of the lead extension 224. The connector 222 includes a connector housing 228. The connector housing 228 defines at least one port 230 into which a proximal end 232 of a lead 234 with terminals 236 can be inserted, as shown by directional arrow 238. The connector housing 228 also includes a plurality of conductive contacts 240. When the lead 234 is inserted into the port 230, the conductive contacts 240 disposed in the connector housing 228 can be aligned with the terminals 236 on the lead 234 to electrically couple the lead extension 224 to the electrodes (134 of FIG. 1) disposed at a distal end (not shown) of the lead 234.

In at least some embodiments, the proximal end of a lead extension is similarly configured and arranged as a proximal end of a lead. The lead extension 224 may include a plurality of conductive wires (not shown) that electrically couple the conductive contacts 240 to a proximal end 248 of the lead extension 224 that is opposite to the distal end 226. In at least some embodiments, the conductive wires disposed in the lead extension 224 can be electrically coupled to a plurality of terminals (not shown) disposed on the proximal end 248 of the lead extension 224. In at least some embodiments, the proximal end 248 of the lead extension 224 is configured and arranged for insertion into a connector disposed in another lead extension. In other embodiments, the proximal end 248 of the lead extension 224 is configured and arranged for insertion into a connector disposed in a control module. As an example, in FIG. 2B the proximal end 248 of the lead extension 224 is inserted into a connector 250 disposed in a control module 252.

It may be useful to design a lead with more electrodes than the leads illustrated in FIGS. 1, 2A, and 2B. For example, a patient may be experiencing pain emanating from an area greater in length than the length of an array of electrodes (e.g., 134 of FIGS. 1 and 2) disposed on the distal end of a conventional lead. For example, a patient may experience pain in an area spanning multiple vertebral bodies. As another example, it may be useful to stimulate two sites separately situated along the spinal cord.

One way to increase stimulation coverage is to provide a lead with a greater length and either increase the amount of space between adjacent electrodes, or increase the size of one or more of the electrodes. When the amount of space between adjacent electrodes is increased or the size of one or more of the electrodes in increased, however, linear electrode density may decrease to a sub-therapeutic level.

Another way to increase stimulation coverage is to provide a lead with a greater length and increase the number of electrodes on the lead. A lead with additional electrodes, however, may also employ an increased number of conductive wires to electrically couple the electrodes to a pulse generator. The increased number of conductive wires may cause the lead to become difficult to electrically couple to the pulse generator. For example, the proximal end of the lead may become incompatible with one or more connectors. As described above, the electrical stimulation system may include one or more connectors disposed on, for example, the control module, the lead extension, an operating room cable, an external trial stimulator, or the like. The proximal end of the lead may be incompatible with the connector for many different reasons. For example, the lateral circumference of the lead may too large to mate with the connector, or the number of terminals disposed on the lead may exceed the number of corresponding connective contacts disposed in the connector. The increased number of conductive wires may also cause the lead to become difficult to implant due to the an increase in the lateral circumference of at least some portions of the lead. The increased lateral circumference of at least some portions of the lead may hinder, or even prevent, insertion of the lead into a patient using a conventional epidural needle.

One option for facilitating compatibility between a lead and a connector is to couple a proximal end of the lead to a lead adaptor that splits the conductive wires at the proximal end of the lead into two or more groupings of conductive wires that each couple with a connector. Lead adapters, however, can be expensive to employ and burdensome to implant in a patient.

In at least some embodiments, stimulation coverage is increased by increasing the number of electrodes disposed at the distal end of a lead without using a lead adaptor to divide conductive wires and without substantially increasing the lateral circumference of a distal end of the lead. In at least some embodiments, the lead includes at least one lead body having a plurality of lead elements, a “multi-lead-element lead body.” The multi-lead-element lead body includes a distal lead element and a plurality of proximal lead elements. In at least some embodiments, the multi-lead-element lead body includes a distal lead element and three proximal lead elements. The distal lead element is coupled to the proximal lead elements via one or more junctions. In at least some embodiments, one or more of the proximal lead elements, in turn, couples to one or more additional lead elements via one or more additional junctions.

Conductive wires extending within the distal lead element are split at the junction into multiple groupings of conductive wires. Each grouping of conductive wires extends within a different proximal lead element. At least one of the proximal lead elements is configured and arranged to couple to a connector such that conductive wires within the proximal element electrically couple to conductive contacts disposed within one or more ports of the connector.

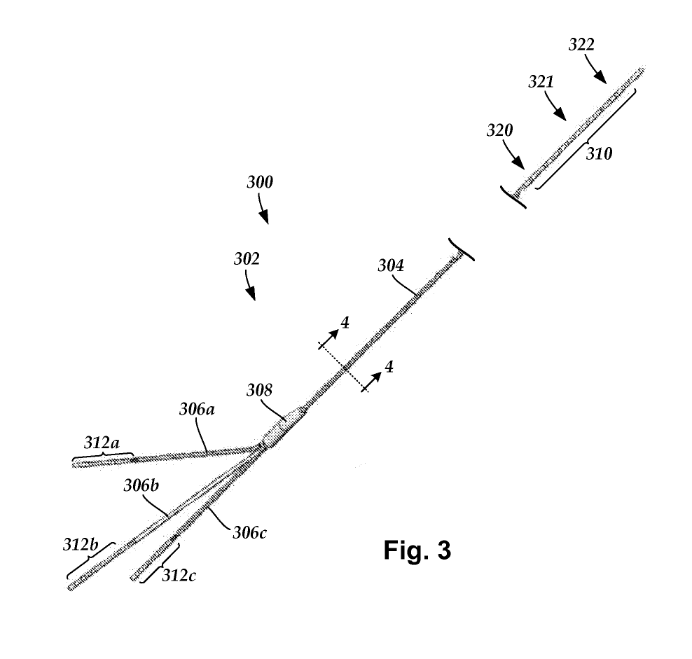

FIG. 3 is a schematic perspective view of one embodiment of a lead 300 that includes a multi-lead-element lead body 302. The multi-lead-element lead body 302 includes a distal lead element 304 and proximal lead elements 306a, 306b, and 306c. The distal lead element 304 is coupled to the proximal lead elements 306a, 306b, and 306c via a junction 308. In at least some embodiments, each of the proximal lead elements 306a, 306b, and 306c are distinct from the distal lead element 304. In at least some other embodiments, the distal lead element 304 extends through the junction 308 as one of the proximal lead elements 306a, 306b, or 306c.

An array of electrodes 310 are disposed on the distal lead element 304. Arrays of terminals 312a, 312b, and 312c are disposed on one or more of the proximal lead elements 306a, 306b, and 306c, respectively. In preferred embodiments, arrays of terminals are disposed on each of the proximal lead elements 306a, 306b, and 306c. In FIG. 3, three proximal lead elements are shown. It will be understood that, in at least some embodiments, other numbers of proximal lead elements may be employed including, for example, four, five, six, seven, eight, nine, ten, or more proximal lead elements.

At least one of the proximal lead elements 306a, 306b, and 306c is configured and arranged for insertion into one or more ports of one or more connectors that are electrically coupleable to one or more pulse generators (e.g., in one or more control modules, external trial stimulators, or the like). In preferred embodiments, each of the proximal lead elements 306a, 306b, and 306c is configured and arranged for insertion into one or more connectors.

In at least some embodiments, one of the proximal lead elements 306a, 306b, or 306c is continuous with the distal lead element 304, while the other of the proximal lead elements 306a, 306b, or 306c couple to the distal lead element 304 via the junction 308. Thus, in at least some embodiments, an exterior portion of the continuous lead element is at least partially removed within the junction 308 to enable access for rerouting conductive wires extending from the electrodes 310 to the non-continuous proximal lead elements. In at least some embodiments, each of the proximal lead elements 306a, 306b, or 306c are non-continuous with the distal lead element 304 and are each coupled to the distal lead element 304 via the junction 308.

The lead elements 304, 306a, 306b, and 306c can be coupled to the junction 308 in any manner. In at least some embodiments, the lead elements 304, 306a, 306b, and 306c are coupled to the junction 308 using a cast junction, a molded-in-place junction, a separate injection molded junction shell to which portions of lead elements 304, 306a, 306b, and 306c are coupled, or extended partially within, or the like. In at least some embodiments, the junction 308 is potted with portions of the lead elements 304, 306a, 306b, and 306c disposed within the junction 308 to provide a joint that is at least one of mechanically secure, electrically isolated, or hydraulically sealed.

Each of the terminal arrays 312a, 312b, and 312c can include any number of terminals. In a preferred embodiment, the collective number of terminals in the terminal arrays 312a, 312b, and 312c is equal to the number of electrodes 708. In preferred embodiments, the terminal arrays 312a, 312b, and 312c each have an equal number of terminals.

Any number of electrodes 310 can be disposed on the distal lead element 304 including, for example, sixteen, seventeen, eighteen nineteen, twenty, twenty-one, twenty-two, twenty-three, twenty-four, twenty-five, twenty-six, twenty-seven, twenty-eight, twenty-nine, thirty, thirty-one, thirty-two, thirty-six, forty, forty-eight, or more electrodes. It will be understood that other numbers of electrodes may be used as well. In at least some embodiments, the electrodes 310 include specific groupings. For example, the electrodes 310 may include a proximal-most group of electrodes 320, one or more middle groups of electrodes 321, or a distal-most group of electrodes 322. In at least some embodiments, the number of groups of electrodes 310 is equal to the number of proximal lead elements.

In at least some embodiments, when an equal number of terminals is disposed on each of the proximal lead elements 306a, 306b, and 306c, the number of electrodes 310 disposed on the distal element 304 is equal to the number of proximal lead elements 306a, 306b, and 306c multiplied by the number of terminals disposed on each of the proximal lead elements 306a, 306b, and 306c. For example, when the multi-lead element lead body 302 has three proximal lead elements, and eight terminals are disposed on each of the of the proximal lead elements, then twenty-four electrodes are disposed on the distal lead element 304.

The conductive wires may be arranged to couple terminals of the terminal arrays 312a, 312b, and 312c to any of the electrodes of the electrode array 310. In at least some embodiments, the conductive wires may be arranged to couple terminals of the terminal arrays 312a, 312b, and 312c to specific electrodes of the electrode array 310. For example, in at least some embodiments terminals of one of the terminal arrays 312a, 312b, and 312c are coupled to the proximal-most group of electrodes 320, while terminals of another of the terminal arrays 312a, 312b, and 312c are coupled to the one or more middle groups of electrodes 321, and terminals of the remaining terminal array are coupled to the distal-most group of electrodes 322.

It will be understood that the terminals of the terminal arrays 312a, 312b, and 312c can be individually coupled to other specific groupings of electrodes. For example, in at least some embodiments, terminals of one or more of the terminal arrays 312a, 312b, and 312c may be coupled to non-adjacent electrodes 310.

Conductive wires can extend along the distal lead element 304 in any manner. As discussed above, with reference to FIG. 1, conductive wires may be embedded in the non-conductive material within the distal lead element 304, or conductive wires can be disposed in one or more lumens extending along the distal lead element 304. In at least some embodiments, each individual conductive wire is disposed in an individual lumen.

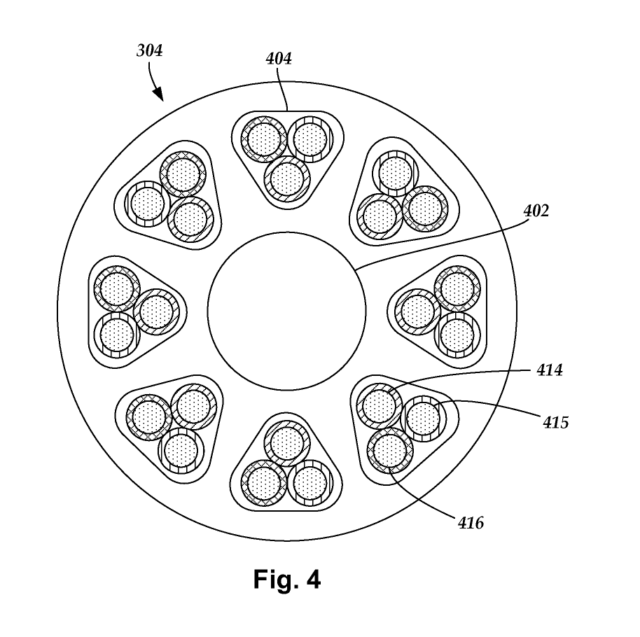

FIG. 4 is a schematic transverse cross-sectional view of one embodiment of the distal lead element 304. In FIG. 4, the distal lead element 304 includes a stylet lumen 402 and a plurality of conductive wire lumens 404. Each conductive wire lumen 404 is configured and arranged to receive multiple conductive wires, such as conductive wires 414-416. In at least some embodiments, the conductive wires 414-416 extend along a length of each individual conductive wire lumen 404 from the electrodes 310 to the junction 308.

In at least some embodiments, the conductive wire lumens 404 each have a triangular transverse cross-sectional profile. It will be understood that the conductive wire lumens 404 can have other transverse cross-sectional profiles including, for example, round, oval, elliptical, rectangular, or the like. It will also be understood that the conductive wire lumens 404 can have irregularly-shaped transverse cross-sectional profiles.

In at least some embodiments, the conductive wire lumens 404 each have a rounded triangular transverse cross-sectional profile. In at least some embodiments, the conductive wire lumens 404 each have a triangular transverse cross-sectional profile with one side of each triangle extending roughly parallel with a closest arced portion of a circumference of the distal lead element 304. In FIG. 4, eight conductive wire lumens 404 are shown, with each conductive wire lumen 404 configured and arranged for receiving three conductive wires 414-416. Thus, in at least some embodiments, twenty-four connector wires 414-416 can be disposed in the conductive wire lumens 404 and electrically coupled individually to twenty-four electrodes 310.

It may be an advantage to form the conductive wire lumens 404 of the distal lead element 304 with a transverse cross-sectional profile that is triangular with one side of each triangle extending roughly parallel with a closest arced portion of a circumference of the distal lead element 304 to enhance electrical isolation between conductive wires. It may also be the case that such an arrangement facilitates steer-ability and response to torquing during insertion of the lead 300 into a patient by increasing wall thicknesses between the conductive wire lumens 404 so that they can support greater loading (during torquing) without deforming or collapsing.

In alternate embodiments, the conductive wire lumens 404 can be configured and arranged to accommodate additional conductive wires 414-416 including, for example, three, four, five, six, seven, eight, nine, ten, twelve, fourteen, sixteen, or more conductive wires 414-416 disposed in each of the conductive wire lumens 404. As will be recognized, other numbers of conductive wires 414-416 may also be disposed in one or more of the conductive wire lumens 404. In at least some embodiments, at least one of the conductive wire lumens 404 is configured and arranged to receive a different number of conductive wires than at least one other of the conductive wire lumens 404.

Conductive wires can extend along the proximal lead elements 306a, 306b, and 306c in any manner. As discussed above, with reference to FIG. 1, conductive wires may be embedded in the non-conductive material within the proximal lead elements 306a, 306b, and 306c, or conductive wires can be disposed in one or more lumens extending along the proximal lead elements 306a, 306b, and 306c. In at least some embodiments, each individual conductive wire is disposed in an individual lumen.

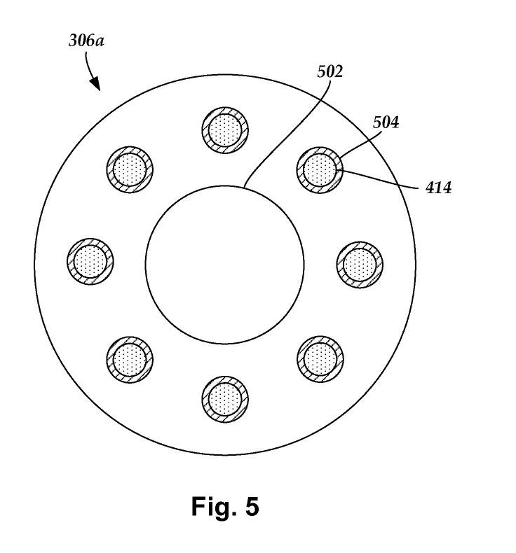

FIG. 5 is a schematic transverse cross-sectional view of one embodiment of one of the proximal lead elements 306a, 306b, and 306c. For simplicity, the proximal lead element of FIG. 5 is referred to as the proximal lead element 306a. In FIG. 5, the proximal lead element 306a includes a stylet lumen 502 and a plurality of conductive wire lumens 504. In at least some embodiments, each conductive wire lumen 504 is configured and arranged for receiving conductive wires 414. In FIG. 5, eight circular-shaped conductive wire lumens 504 are shown. Thus, in the proximal lead element 306a shown in FIG. 4, eight connector wires 414 can be disposed in the conductive wire lumens 504 and electrically coupled to eight terminals 312a.

In at least some embodiments, only one or two of the proximal lead elements 306a, 306b, and 306c has the transverse cross-sectional profile shown in FIG. 5. In at least some embodiments, each of the proximal lead elements 306a, 306b, and 306c has the transverse cross-sectional profile shown in FIG. 5.

In at least some embodiments, the stylet lumen 502 of the proximal lead element 306a connects with the stylet lumen 402 of the distal lead element 304 in the junction 308. In at least some embodiments, the stylet lumen 502 of the proximal lead element 306a connects with the stylet lumen 402 of the distal lead element 304 such that a stylet can be inserted into the stylet lumen 502 of the proximal lead element 306a and be extended into the stylet lumen 402 of the distal lead element 304. In at least some embodiments, at least one of the other proximal lead elements 306b or 306c also has a stylet lumen through which a stylet can be extended into the stylet lumen 402. Thus, in at least some embodiments, at least two stylets may be inserted into the lead 300. In at least some embodiments, each of the proximal lead elements 306a, 306b, 306c define stylet lumens, yet the stylet lumens of at least one of the proximal lead elements 306b and 306c is plugged.

The distal and proximal lead elements can be any length. In preferred embodiments, the distal lead element 304 is longer than the proximal lead elements 306a, 306b, and 306c. In at least some embodiments, however, the proximal lead elements 306a, 306b, and 306c are at least as long as the distal lead element 304. In at least some embodiments, at least two of the proximal lead elements 306a, 306b, and 306c have different lengths from one another. In at least some embodiments, each of the proximal lead elements 306a, 306b, and 306c have different visible lengths from one another.

In at least some embodiments, the multi-lead element lead body 302 includes an identification mechanism. In at least some embodiments, the identification mechanism facilitates lead fabrication. In at least some embodiments, the conductive wires 414-416 extending through the conductive wire lumens 404 are distinguishable from one another, for example, to facilitate management of the conductive wires 414-416 during loading of the conductive wires 414-416 into the distal lead element 304, or during coupling of the conductive wires 414-416 to the electrodes 310. In at least some embodiments a visually or texturally distinct insulation is individually disposed around each of the conductive wires 414-416. In at least some embodiments, each of conductive wires 414-416 is coated with insulation having a different color than the insulation coating the other remaining conductive wires 414-416. In at least some embodiments, each of conductive wires 414-416 is coated with insulation having a different color pattern than the insulation coating of the other remaining conductive wires 414-416.

In at least some embodiments, the identification mechanism facilitates identifying which electrodes on the electrode array 310 are coupled to terminals disposed on which of the terminal arrays 312a, 312b, and 312c. As discussed above, in at least some embodiments each of the proximal lead elements 306a, 306b, and 306c has a different length. In at least some embodiments, the length of each proximal lead element 306a, 306b, and 306c corresponds to a particular group of electrodes of the electrode array 310. For example, the terminals disposed on the longest of the proximal lead elements 306a, 306b, and 306c may couple to the proximal-most or the distal-most electrodes of the electrode array 310, while another of the proximal lead elements 306a, 306b, and 306c may couple to the other of the proximal-most or the distal-most electrodes of the electrode array 310.

The distal lead element 304 can have any diameter suitable for percutaneous implantation into a patient. In at least some embodiments, the distal lead element 304 has a larger diameter than at least one of the proximal lead elements 306a, 306b, and 306c. In at least some embodiments, the distal lead element 304 has a diameter no larger than 0.08 inches (roughly 0.2 cm), 0.075 inches (roughly 0.2 cm), 0.07 inches (roughly 0.2 cm), 0.065 inches (roughly 0.2 cm), 0.06 inches (roughly 0.2 cm), 0.055 inches (roughly 0.1 cm), or 0.05 inches (roughly 0.1 cm).

In at least some embodiments, the proximal lead elements 306a, 306b, and 306c also have diameters suitable for percutaneous implantation into a patient. In at least some embodiments, the proximal lead elements 306a, 306b, and 306c have equal diameters. In at least some embodiments, the proximal lead elements 306a, 306b, and 306c have diameters no larger than 0.065 inches (roughly 0.2 cm), 0.060 inches (roughly 0.1 cm), 0.055 inches (roughly 0.1 cm), or 0.05 inches (roughly 0.1 cm). In at least some embodiments, the diameter of the distal lead element 304 is no more than 0.015 inches (roughly 0.38 mm), 0.01 inches (roughly 0.25 mm), or 0.005 inches (roughly 0.13 mm) larger than the individual diameter of at least one of the proximal lead elements 306a, 306b, and 306c.

At least some lead bodies are isodiametric to facilitate sliding of an epidural needle over a proximal end of a lead during removal of the epidural needle from a patient once the lead is positioned within a patient. In at least some embodiments, the junction 308 has a circumference that is larger than a circumference of the distal lead element 304. In at least some embodiments, a collective circumference of the proximal lead elements 306a, 306b, and 306c is larger than a circumference of the distal lead element 304. Thus, the larger-sized portions of the lead 300 may hinder, or even prevent, a conventional epidural needle from sliding off the proximal end of the lead 300.

In at least some embodiments, a kit for providing electrical stimulation of patient tissue includes the lead 300 and a lead introducer configured and arranged for facilitating percutaneous insertion of the lead 300 into a patient. In at least some embodiments, the lead introducer is a lateral-release lead introducer (“lead introducer”) which uses a multi-piece insertion needle that enables the lead 300 to be laterally separated from the multi-piece insertion needle. In at least some embodiments, the lead 300 may be laterally separated from the multi-piece insertion needle without sliding the multi-piece insertion needle off the proximal end of the lead 300. In at least some embodiments, the lead 300 laterally separates from the multi-piece insertion needle by passing the lead 300 through an open channel defined along a length of the multi-piece insertion needle. In at least some embodiments, during implantation of the lead 300 the multi-piece insertion needle is disposed in a splitable member that separates from the lead by splitting apart along a length of the splitable member.

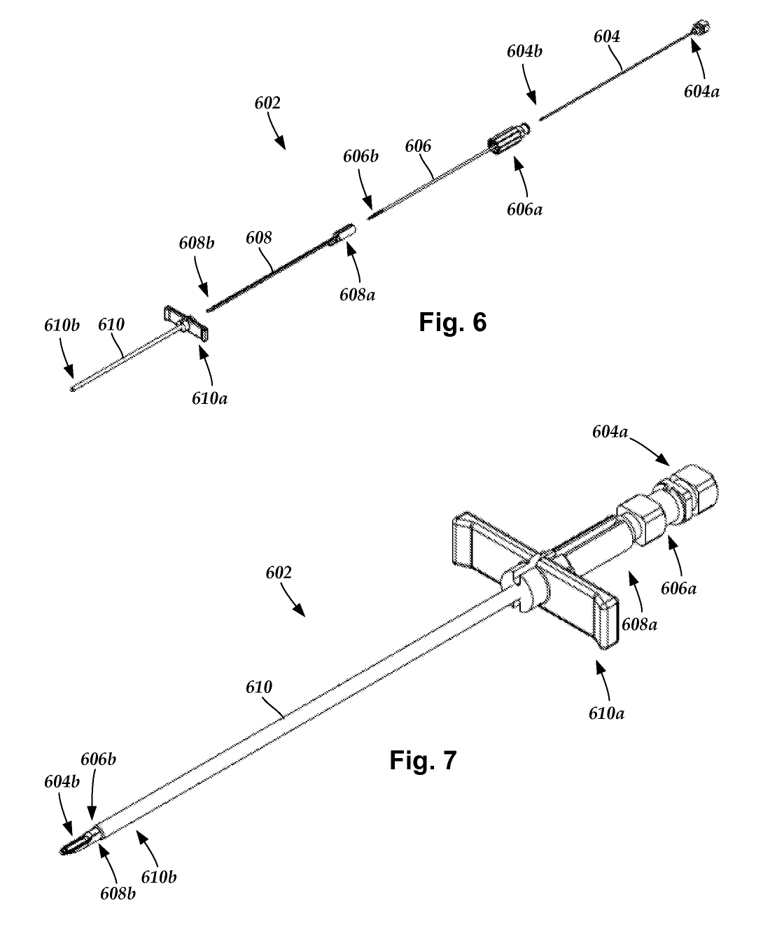

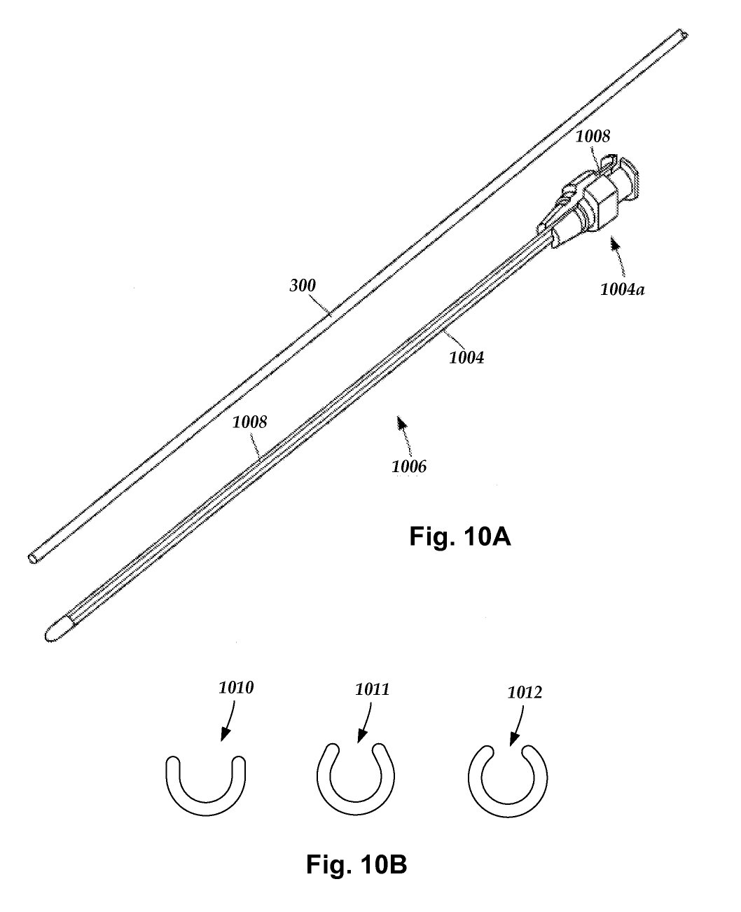

FIG. 6 is a schematic perspective exploded view of one embodiment of a lead introducer 602 configured and arranged to facilitate implantation of an electrical stimulation system into a patient. The lead introducer 602 includes an obturator 604, an inner insertion needle 606, an outer insertion needle 608, and a splitable member 610. The obturator 604 has a proximal hub 604a and a distal end 604b. The inner insertion needle 606 has a proximal hub 606a and a distal end 606b, and defines a lumen (not shown) extending from the proximal hub 606a. The outer insertion needle 608 has a proximal hub 608a and a distal end 608b, and defines an open channel (1004 in FIG. 10) extending along a length of the outer insertion needle 608. The splitable member 610 has a proximal hub 610a and a distal end 610b, and defines a lumen (not shown) extending from the proximal hub 610a.

In at least some embodiments, the distal end 604b of the obturator 604 is configured and arranged for insertion into the lumen of the inner insertion needle 606. In at least some embodiment, the distal end 606b of the inner insertion needle 606 is configured and arranged for insertion into the open channel (1004 in FIG. 10) of the outer insertion needle 608. In at least some embodiments, the distal end 608b of the outer insertion needle 608 is configured and arranged for insertion into the lumen of the splitable member 610.

In at least some embodiments, the obturator 604, the inner insertion needle 606, the outer insertion needle 608, and the splitable member 610 are coupleable to one another such that the obturator 604, the inner insertion needle 606, the outer insertion needle 608, and the splitable member 610 form a nested arrangement. FIG. 7 is a schematic perspective view of one embodiment of the obturator 604 disposed in the inner insertion needle 606 which, in turn, is disposed in the outer insertion needle 608 which, in turn is disposed in the splitable member 610.

In at least some embodiments, the obturator 604, the inner insertion needle 606, the outer insertion needle 608, and the splitable member 610 are coupleable to one another such that the proximal hubs 604a, 606a, 608a, and 610a of the obturator 604, the inner insertion needle 606, the outer insertion needle 608, and the splitable member 610, respectively, align axially to one another. In at least some embodiments, the obturator 604, the inner insertion needle 606, the outer insertion needle 608, and the splitable member 610 are coupleable to one another such that the distal ends 604b, 606b, and 608b of the obturator 604, the inner insertion needle 606, and the outer insertion needle 608, respectively, extend distally beyond the distal end 610b of the splitable member 610.

In at least some embodiments, the outer insertion needle 608 is formed from a rigid material suitable for implantation, such as stainless steel. In at least some embodiments, the inner insertion needle 606 is formed from the same material as the outer insertion needle 608. In at least some embodiments, the inner insertion needle 606 is formed from a material that is more flexible than the outer insertion needle 608. In at least some embodiments, the outer insertion needle 608 is formed from a material that is more rigid than the splitable member 610. In at least some embodiments, the outer insertion needle 608 is formed from a material that is rigid enough to enable the outer insertion needle 608 to be used to guide (e.g., enable lateral steering) the splitable member 610 within a patient when the outer insertion needle 608 is disposed in the splitable member 610.

In at least some embodiments, the lateral circumference of the outer insertion needle 608 is no greater than eighteen-gauge. In at least some embodiments, the lateral circumference of the outer insertion needle 608 is no greater than seventeen-gauge. In at least some embodiments, the lateral circumference of the outer insertion needle 608 is no greater than sixteen-gauge. In at least some embodiments, the lateral circumference of the outer insertion needle 608 is no greater than fifteen-gauge. In at least some embodiments, the lateral circumference of the outer insertion needle 608 is no greater than fourteen-gauge. In at least some embodiments, the lateral circumference of the outer insertion needle 608 is no greater than thirteen-gauge. In at least some embodiments, the lateral circumference of the outer insertion needle 608 is no greater than twelve-gauge.

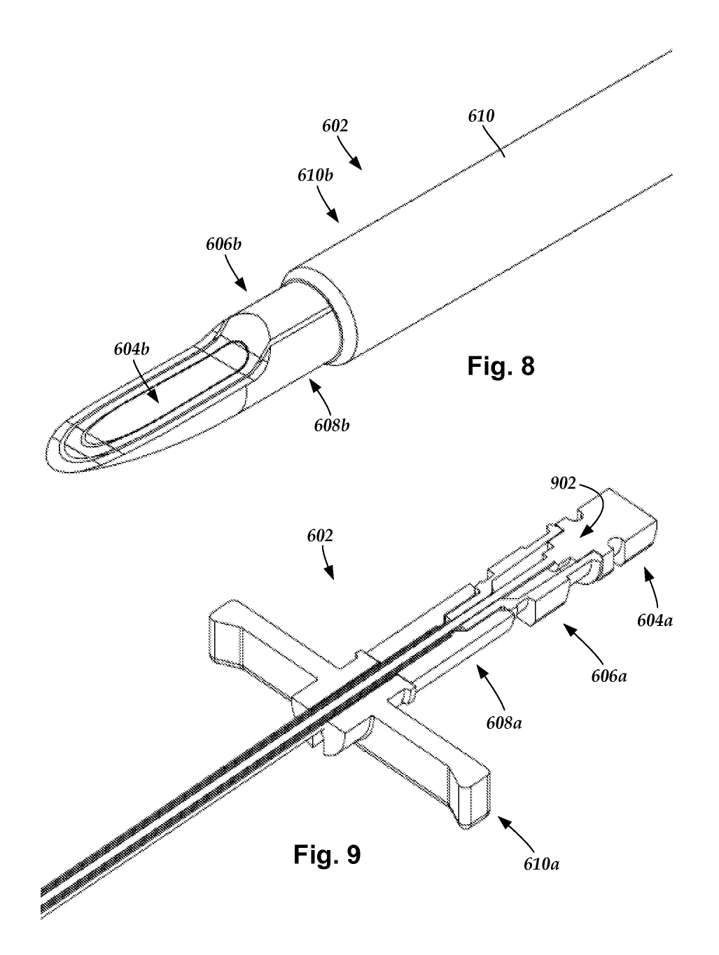

FIG. 8 is a schematic perspective close-up view of one embodiment of a distal end of the lead introducer 602. In at least some embodiments, the obturator 604, the inner insertion needle 606, the outer insertion needle 608, and the splitable member 610 coupleable to one another such that the distal ends 604b, 606b, and 608b of the obturator 604, the inner insertion needle 606, and the outer insertion needle 608, respectively, extend distally beyond the distal end 610b of the splitable member 610. In FIG. 8, the distal tips of the distal ends 604b and 606b of the obturator 604 and the inner insertion needle 606 are removed, for clarity of illustration. In at least some embodiments, the distal end 604b of the obturator 604 includes a blunt tip to reduce or prevent coring of patient tissue during insertion of the lead introducer 602 into a patient.

FIG. 9 is a schematic perspective longitudinal cross-sectional view of one embodiment of a proximal end of the lead introducer 602. In FIG. 9, the obturator 604, the inner insertion needle 606, the outer insertion needle 608, and the splitable member 610 are coupleable to one another such that the proximal hubs 604a, 606a, 608a, and 610a of the obturator 604, the inner insertion needle 606, the outer insertion needle 608, and the splitable member 610, respectively, are axially aligned to one another.

In at least some embodiments, the proximal hub 606a of the inner insertion needle includes a luer fitting 902 configured and arranged to receive a syringe. In at least some embodiments, fluid (e.g., saline solution, air, or the like) may be introduced or removed through the luer fitting 902 to check for precise positioning of the lead introducer 602, for example, whether or not the epidural space has been entered.

In at least some embodiments, the outer insertion needle 608 is configured and arranged to receive a distal end of a lead when the inner insertion needle 606 is not disposed in the outer insertion needle 608. FIG. 10A is a schematic perspective view of one embodiment of a distal end of a lead 300 and the outer insertion needle 608 of the lead introducer 602. The outer insertion needle 608 defines an open channel 1004 that extends substantially entirely along a length of the outer insertion needle 608 and that is configured and arranged to receive the distal end of the lead 300. In at least some embodiments, the open channel 1004 extends along the proximal hub 610a of the outer insertion needle 608. In at least some embodiments, the open channel 1004 extends along the entire length of the of the outer insertion needle 608.

In some embodiments, the lead 300 has an isodiametric lead body. In other embodiments, the lead 300 has a non-isodiametric lead body. In at least some embodiments, the lead 300 includes one or more elements (e.g., a junction, adaptor, or the like) disposed along the length of the lead 300 which have a transverse cross-sectional shape or size that is different from at least one other portion of the lead 300. In at least some embodiments, the distal end of the lead 300 has a transverse cross-sectional shape that is similar to the inner insertion needle 606. In at least some embodiments, the one or more elements having a different transverse cross-sectional shape or size are disposed on a proximal end of the lead 300.

In at least some embodiments, the inner insertion needle 606 is retained in the open channel 1004 by the splitable member 610. In at least some embodiments, the inner insertion needle 606 is configured and arranged to at least substantially fill the open channel 1004 when the inner insertion needle 606 is disposed in the open channel 1004. In at least some embodiments, the inner insertion needle 606 is configured and arranged for insertion into and out of the open channel 1004 of the outer insertion needle 608 by sliding the inner insertion needle 606 axially along the open channel 1004.

In at least some embodiments, the open channel 1004 is configured and arranged to receive the lead 300 when the inner insertion needle 606 is not disposed in the open channel 1004. In at least some embodiments, the open channel 1004 has a width that is no less than a diameter of the lead 300. In at least some embodiments, the open channel 1004 is configured and arranged to receive the lead 300 such that the lead 300 may be separated from the open channel 1004 without moving the lead 300 axially relative to the outer insertion needle 608.

In at least some embodiments, the lead 300 may be removed from the open channel 1004 by applying enough lateral force to at least one of the lead 300 or the outer insertion needle 608 to pass the lead 300 out through the open channel 1004. In at least some embodiments, the open channel 1004 is configured and arranged such that, when the splitable member 610 is removed from the outer insertion needle 608, the lead 300 separates from the outer insertion needle 608 without needing to apply lateral force to either the lead 300 or the outer insertion needle 608.

FIG. 10B is a schematic transverse cross-sectional view of several different exemplary embodiments of the open channel 1004. In at least some embodiments, the portions of the outer insertion needle 608 along which the open channel 1004 extends have a transverse cross-sectional shape that is at least substantially U-shaped 1010. In at least some embodiments, the portions of the outer insertion needle 608 along which the open channel 1004 extends have a transverse cross-sectional shape that is at least substantially horseshoe-shaped 1011. In at least some embodiments, the portions of the outer insertion needle 608 along which the open channel 1004 extends have a transverse cross-sectional shape that is at least substantially C-shaped 1012.

In at least some embodiments, the lead 300 may be inserted into a patient using the lead introducer 602. In at least some embodiments, the obturator 604 is inserted into the lumen of the inner insertion needle 606, the inner insertion needle 606 is inserted into the open channel 1004 of the outer insertion needle 608, and the outer insertion needle 608 is inserted into the splitable member 610, as shown in FIG. 6 and FIG. 7. It will be understood that the components of the lead introducer 602 can be assembled in any order. For example, the inner insertion needle 606 can be inserted into the open channel 1004 of the outer insertion needle 608 either before or after the outer insertion needle 608 is inserted into the splitable member 610. In preferred embodiments, the lead introducer 602 is pre-assembled prior to insertion into the patient. In preferred embodiments, the lead introducer 602 is pre-assembled prior to a procedure to insert the lead introducer 602 into the patient.

The assembled lead introducer 602 is inserted into a patient and guided in proximity to a target stimulation location (e.g., several vertebrae levels above or below the target stimulation location. In at least some embodiments, once the lead introducer 602 is in proximity to a target stimulation location, the obturator 604 is removed and fluid is introduced or removed through the luer fitting 902 of the inner insertion needle 606 to check for precise positioning of the lead introducer 602, for example, in an epidural space of the patient.

FIG. 11 is a schematic perspective view of one embodiment of the inner insertion needle 606 inserted into the open channel 1004 of the outer insertion needle 608 which, in turn, is inserted into the splitable member 610. In FIG. 11, the inner insertion needle 606, the outer insertion needle 608, and the splitable member 610 are disposed in a patient, as shown by a dotted line 1102. The obturator 604 has been removed to allow access to the luer fitting 902 for performing the loss of resistance test.

It will be understood that the assembled lead introducer 602 can also be inserted into a patient and guided in proximity to a target stimulation location without using the obturator 604.

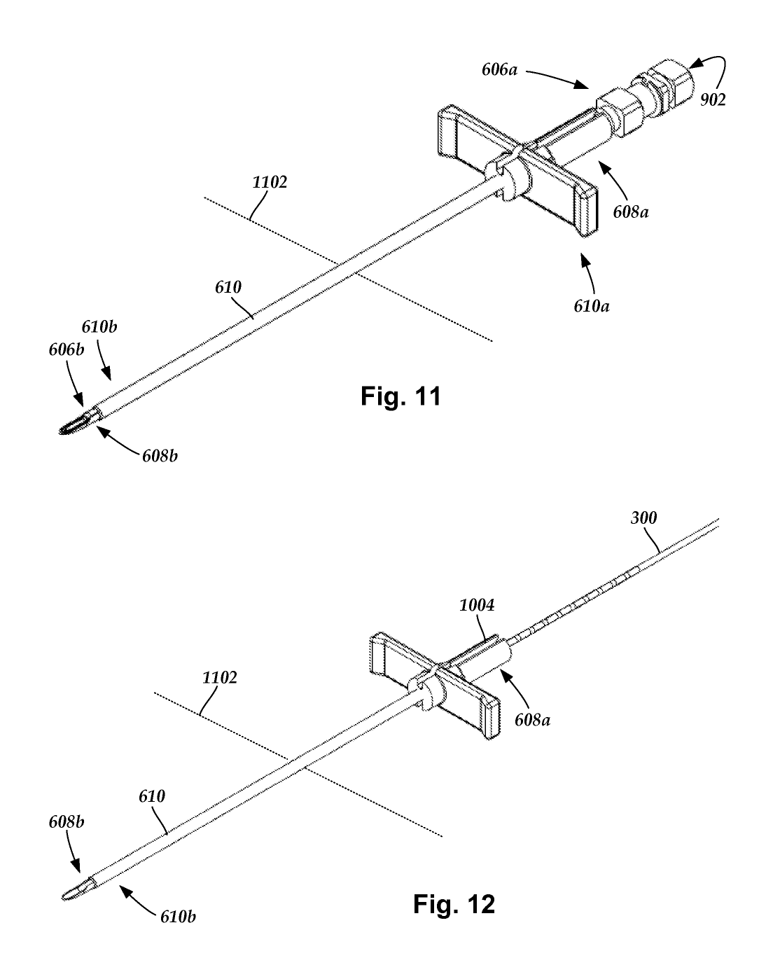

Once the lead introducer 602 is positioned in the epidural space in proximity to the target stimulation location, the inner insertion needle 606 may be removed and the distal end of the lead 300 may be inserted into the open channel 1004 of the outer insertion needle 608. FIG. 12 is a schematic perspective view of one embodiment of the distal end of the lead 300 inserted into the open channel 1004 of the outer insertion needle 608 via the proximal hub 608a. Once the distal end of the lead 300 is inserted into the open channel 1004 of the outer insertion needle 608, the distal end of the lead 300 is guided to the target stimulation region. In at least some embodiments, the distal end of the lead 300 is guided to the target stimulation region by the comparably rigid outer insertion needle 608.

It may be an advantage to guide the lead 300 within the patient while the lead 300 is disposed in the outer insertion needle 608 and the splitable member 610. The outer insertion needle 608 and the splitable member 610 may provide the clinician with the ability to steer the lead introducer 602 by applying a laterally force of the lead introducer 602 to direct the trajectory of the lead 300. When the outer insertion needle 608 is removed from the lead 300 prior to insertion, then the splitable member 610 may be too flexible to provide this steering ability.

Once the distal end of the lead 300 has been guided to the target stimulation location, the splitable member 610 and the outer insertion needle 608 may be separated from the lead 300 and removed from the patient. It will be understood that the splitable member 610 may be separated from the lead 300 either before or after the outer insertion needle 608 is separated from the lead 300. It will also be understood that the splitable member 610 may be removed from the patient either before or after the outer insertion needle 608 is removed from the patient. In some embodiments, the outer insertion needle 608 is separated from the lead 300 prior to the splitable member 610 being separated from the lead 300. In other embodiments, the splitable member 610 is separated from the lead 300 prior to the outer insertion needle 608 being separated from the lead 300. In some embodiments, the outer insertion needle 608 is removed from the patient prior to removal of the splitable member 610. In other embodiments, the splitable member 610 is removed from the patient prior to removal of the outer insertion needle 608.

In preferred embodiments, the lead 300 is guided to the target stimulation location while disposed in the outer insertion needle 608 and the splitable member 610. The outer insertion needle 608 is removed from the lead 300 (and from the patient). The splitable member 610 is then split apart from the lead 300 and removed from the patient.

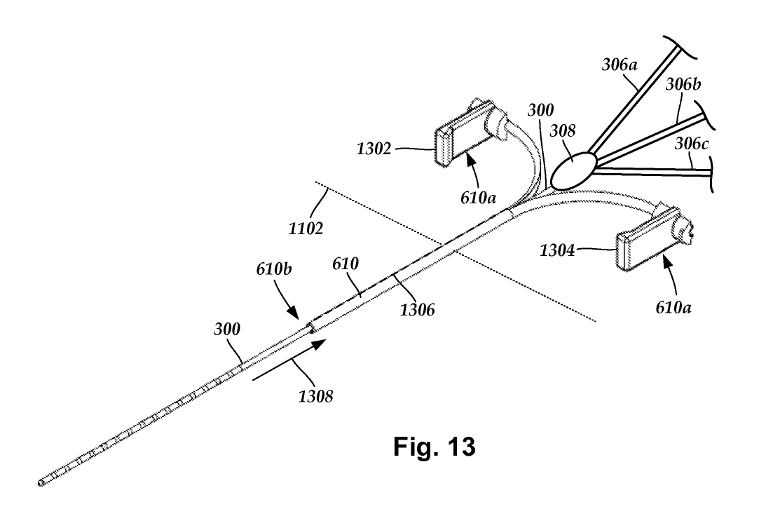

FIG. 13 is a schematic perspective view of one embodiment of the splitable member 610 being split apart to remove the splitable member 610 from the lead 300. The proximal hub 610a of the splitable member 610 includes at least two pull-apart tabs 1302 and 1304.

In at least some embodiments, the splitable member 610 is formed from a flexible material suitable for implantation into the patient 1102 including, for example, fluorinated ethylene propylene, polytetrafluoroethylene, high-density polyethylene, polyetheretherketone, and the like or combinations thereof. Additionally, one or more radiopaque materials may be added including, for example, barium sulfate and bismuth subcarbonate, and the like or combinations thereof to facilitate implantation of the introducer sheath through the use of one or more medical imaging techniques, such as fluoroscopy.

In at least some embodiments, the splitable member includes one or more weakened regions 1306, such as score lines or perforations, extending along at least a portion of a length of the splitable member 610 from between the at least two pull-apart tabs 1002 and 1004. In at least some embodiments, when the at least two pull-apart tabs 1302 and 1304 are separated from one another, for example, by pulling each pull-apart tab away from the other pull-apart tab(s) in directions approximately orthogonal to the splitable member 610, the splitable member 610 separates along the one or more weakened regions 1306.

In at least some embodiments, the splitable member 610 is separated into a plurality of longitudinal strips while pulling the splitable member 610 proximally along the lead 300. As the splitable member 610 splits apart, the distal end 610b of the splitable member 610 moves proximally along the lead 300 (as shown by arrow 1308), with an increasing amount of the lead 300 extending through the distal end 610b of the splitable member 610. In at least some embodiments, an undersurface of the splitable member 610 includes a lubricious coating to facilitate the proximal movement of the splitable member 610.

Eventually, the splitable member 610 may be completely separated into two or more longitudinal strips, thereby separating completely from the lead 300 and also from the patient. In at least some embodiments, the distal ends of the splitable member 610 may be extracted from the patient as the splitable member 610 is split apart. In at least some embodiments, the splitable member 610 may be split apart without causing the lead 300 to move.

Once the lead 300 is positioned at the target stimulation site, the lead 300 may be coupled to one or more control modules (e.g., 102 of FIG. 1) and implanted using well-known techniques, for example, using one or more using tunneling straws placed in passageways underneath patient skin with bores that are sized large enough to receive the lead 300. In at least some embodiments, the lead 300 may be coupled to one or more connectors of one or more control modules, as shown in FIGS. 2A-2B. In other embodiments, the lead 300 may be coupled to one or more other devices, including an adaptor, a lead extension, an operating room cable, or the like or combinations thereof.

It will be understood that other types of lead introducers may be used as well, including, for example, a splitable lead introducer that uses a split-release insertion needle. In at least some embodiments, the lead introducer includes a split-release insertion needle formed from a plurality of body elements and a removable retaining member, such as heat shrink tubing, disposed over at least a portion of the split-release insertion needle. When the retaining member is removed from the split-release insertion needle, the body elements at least partially separate from one another, thereby enabling the body elements to separate from the lead 300. In at least some embodiments, when the retaining member is removed from the split-release insertion needle, the body elements completely detach from one another.

In at least some embodiments, the body elements at least partially separate from one another along a longitudinal axis of the split-release insertion needle. In at least some embodiments, the body elements separate from one another such that at least some of the plurality of body elements remain coupled together. In at least some embodiments, the body elements separate from one another such that at least some of the body elements completely detach from one another. When the body elements are separated (either partially or fully) from one another, the body elements may be removed from the patient, leaving the lead 300 in place. In at least some embodiments, when the body elements are separated (either partially or fully) from one another, the body elements may be removed from the patient without sliding the split-release insertion needle off the proximal end of the lead 300 through the lumen of the lead introducer.

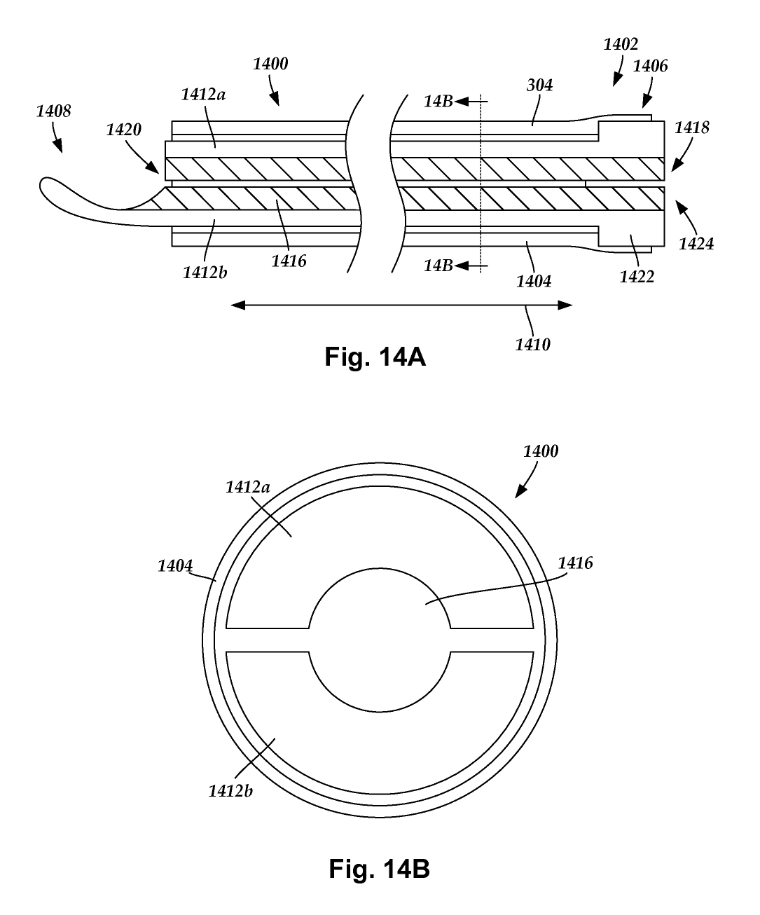

FIG. 14A is a schematic longitudinal cross-sectional view of one embodiment of a lead introducer 1400 that includes a split-release insertion needle 1402 and a removable retaining member 1404 disposed over the split-release insertion needle 1402. It will be understood that the components of FIG. 14A are not drawn to scale. For example, the thickness of the remaining member 1404 is shown with an exaggerated thickness, for clarity of illustration.

FIG. 14B is a schematic transverse cross-sectional view of the lead introducer 1400. The split-release insertion needle 1402 includes a proximal end 1406, a distal end 1408, and a longitudinal axis 1410 (shown by a two-headed arrow). The split-release insertion needle 1402 also includes a plurality of body elements 1412a and 1412b mated together to define a lumen 1416 configured and arranged to receive the lead 300. In at least some embodiments, the body elements 1412a and 1412b are mated along the longitudinal axis 1410 of the split-release insertion needle 1402. In at least some embodiments, the lumen 1416 extends along the longitudinal axis 1410. In at least some embodiments, the lumen 1416 extends along the longitudinal axis 1410 from the proximal end 1406 to the distal end 1408 of the split-release insertion needle 1402. In at least some embodiments, the lumen 1416 extends from a proximal aperture 1418 at the proximal end 1406. In at least some embodiments, the lumen 1416 extends from a distal aperture 1420 at the distal end 1408.

In at least some embodiments, the proximal end 1408 includes a proximal hub 1422. In at least some embodiments, the lumen 1416 is in fluid communication with a luer fitting 1424. In at least some embodiments, the luer fitting 1424 is disposed on the proximal hub 1422. In at least some embodiments, the luer fitting 1424 is configured and arranged to receive a syringe. In at least some embodiments, fluid (e.g., saline solution, air, or the like) may then be introduced or removed through the luer fitting 1424 to check for precise positioning of the lead introducer 1400, for example, in an epidural space of the patient.

The retaining member 1404 may be formed from any thermoplastic material suitable for implantation including, for example, polyester, polyolefin, one or more fluoropolymers (such as fluorinated ethylene propylene, polytetrafluoroethylene, polyvinylidene fluoride, or the like or combinations thereof), polyvinyl chloride, polychloroprene, silicone elastomer, or the like or combinations thereof.

In at least some embodiments, the retaining member 1404 is disposed over at least a portion of an outer surface of the split-release insertion needle 1402. In at least some embodiments, the retaining member 1404 is disposed substantially entirely over the outer surface of the split-release insertion needle 1402 distal to the proximal hub 1422. In at least some embodiments, the retaining member 1404 is disposed entirely over the outer surface of the split-release insertion needle 1402. In at least some embodiments, the retaining member 1404 forms a watertight seal along the lumen 1416 of the split-release insertion needle 1402. In at least some embodiments, the retaining member 1404 provides a pressure-tight seal for the lumen 1416. Providing the pressure-tight seal may enable a loss-of-resistance technique to be performed to ensure entry into the epidural space, as mentioned above.

The body elements 1412a and 1412b of the split-release insertion needle 1402 are formed from one or more rigid materials suitable for implantation (e.g., one or more metals, alloys, rigid plastics, or the like). In some embodiments, each of the body elements 1412a and 1412b are formed from the same material(s). In other embodiments, at least one of the body elements 1412a and 1412b is formed from one or more materials that are different from at least another one of the body elements 1412a and 1412b. The split-release insertion needle 1402 is formed from a material that is rigid enough to enable the split-release insertion needle 1402 to be used to guide the lead introducer 1400 within a patient.

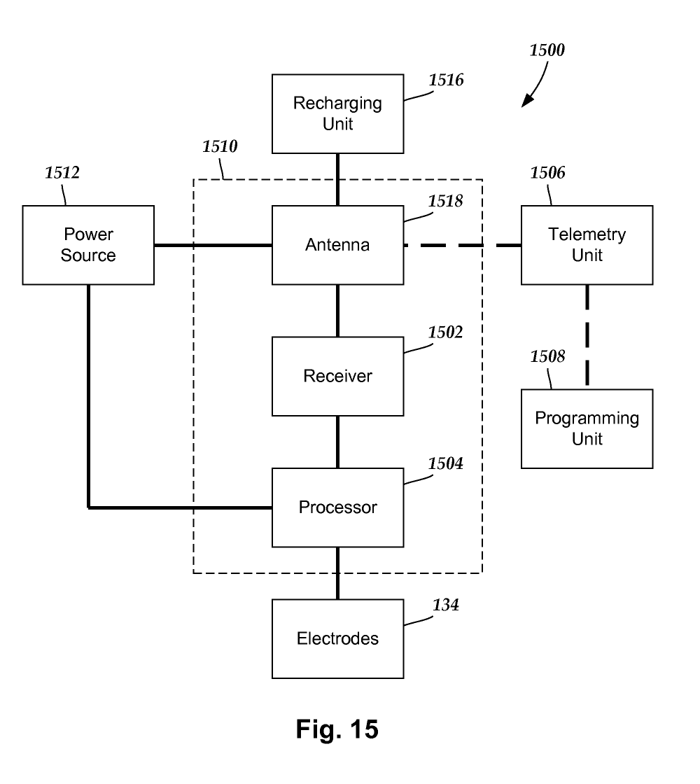

FIG. 15 is a schematic overview of one embodiment of components of an electrical stimulation system 1500 including an electronic subassembly 1510 disposed within a control module. It will be understood that the electrical stimulation system can include more, fewer, or different components and can have a variety of different configurations including those configurations disclosed in the stimulator references cited herein.

Some of the components (for example, power source 1512, antenna 1518, receiver 1502, and processor 1504) of the electrical stimulation system can be positioned on one or more circuit boards or similar carriers within a sealed housing of an implantable pulse generator, if desired. Any power source 1512 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Patent Application Publication No. 2004/0059392, incorporated herein by reference.

As another alternative, power can be supplied by an external power source through inductive coupling via the optional antenna 1518 or a secondary antenna. The external power source can be in a device that is mounted on the skin of the user or in a unit that is provided near the user on a permanent or periodic basis.

If the power source 1512 is a rechargeable battery, the battery may be recharged using the optional antenna 1518, if desired. Power can be provided to the battery for recharging by inductively coupling the battery through the antenna to a recharging unit 1516 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1504 is generally included to control the timing and electrical characteristics of the electrical stimulation system. For example, the processor 1504 can, if desired, control one or more of the timing, frequency, strength, duration, and waveform of the pulses. In addition, the processor 1504 can select which electrodes can be used to provide stimulation, if desired. In some embodiments, the processor 1504 may select which electrode(s) are cathodes and which electrode(s) are anodes. In some embodiments, the processor 1504 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1508 that, for example, allows modification of pulse characteristics. In the illustrated embodiment, the processor 1504 is coupled to a receiver 1502 which, in turn, is coupled to the optional antenna 1518. This allows the processor 1504 to receive instructions from an external source to, for example, direct the pulse characteristics and the selection of electrodes, if desired.

In one embodiment, the antenna 1518 is capable of receiving signals (e.g., RF signals) from an external telemetry unit 1506 which is programmed by a programming unit 1508. The programming unit 1508 can be external to, or part of, the telemetry unit 1506. The telemetry unit 1506 can be a device that is worn on the skin of the user or can be carried by the user and can have a form similar to a pager, cellular phone, or remote control, if desired. As another alternative, the telemetry unit 1506 may not be worn or carried by the user but may only be available at a home station or at a clinician's office. The programming unit 1508 can be any unit that can provide information to the telemetry unit 1506 for transmission to the electrical stimulation system 1500. The programming unit 1508 can be part of the telemetry unit 1506 or can provide signals or information to the telemetry unit 1506 via a wireless or wired connection. One example of a suitable programming unit is a computer operated by the user or clinician to send signals to the telemetry unit 1506.

The signals sent to the processor 1504 via the antenna 1518 and receiver 1502 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1500 to cease operation, to start operation, to start charging the battery, or to stop charging the battery. In other embodiments, the stimulation system does not include an antenna 1518 or receiver 1502 and the processor 1504 operates as programmed.

Optionally, the electrical stimulation system 1500 may include a transmitter (not shown) coupled to the processor 1504 and the antenna 1518 for transmitting signals back to the telemetry unit 1506 or another unit capable of receiving the signals. For example, the electrical stimulation system 1500 may transmit signals indicating whether the electrical stimulation system 1500 is operating properly or not or indicating when the battery needs to be charged or the level of charge remaining in the battery. The processor 1504 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A lead for providing electrical stimulation of patient tissue, the lead comprising:
a distal lead element having a longitudinal axis, the distal lead element comprising
at least twenty electrodes disposed on the distal lead element,
a stylet lumen extending along at least a portion of the longitudinal axis of the distal lead element, and
a plurality of conductive wire lumens extending along at least a portion of the longitudinal axis of the distal lead element;
three proximal lead elements, each of the three proximal lead elements having a longitudinal axis and comprising
a plurality of terminals disposed on the proximal lead element, and
a plurality of conductive wire lumens extending along at least a portion of the longitudinal axis of the proximal lead element, wherein each of the conductive wire lumens has a round transverse cross-sectional shape;
a junction coupling the distal lead element to the proximal lead elements; and
a plurality of conductive wires coupling each of the plurality of electrodes disposed on the distal lead element to at least one of the plurality of terminals disposed on at least one of the proximal lead elements;
wherein the plurality of conductive wire lumens disposed on the distal lead element are configured and arranged to receive a plurality of the conductive wires.

2. The lead of claim 1, wherein each of the plurality of conductive wire lumens disposed on the distal lead element is configured and arranged to receive three of the conductive wires.

3. The lead of claim 2, wherein each of the three conductive wires extending along a first one of the plurality of conductive wire lumens of the distal lead element couples to one or more terminals disposed on a different one of the three proximal lead elements.

4. The lead of claim 1, wherein for each of the plurality of conductive wires extending along a first one of the plurality of conductive wire lumens of the distal lead element, insulation is disposed around the conductive wire that is visually or texturally distinct from insulation disposed around the remaining conductive wires disposed in the first one of the plurality of conductive wire lumens.

5. The lead of claim 1, wherein each of the proximal lead elements has a visibly different length.

6. The lead of claim 1, wherein for each proximal lead element the length of the proximal lead element correlates to which electrodes the terminals of the proximal lead element are coupled.

7. The lead of claim 1, wherein one of the proximal lead elements is continuous with the distal lead element.

8. An electrical stimulating system comprising:
the lead of claim 1;
at least one control module configured and arranged to electrically couple to each of the proximal lead elements, each of the at least one control module comprising
a housing, and
an electronic subassembly disposed in the housing; and
a connector for receiving at least one of the three proximal lead elements, the connector comprising
a connector housing defining at least one port at a distal end of the connector, the at least one port configured and arranged for receiving at least one of the three proximal lead elements, and
a plurality of connector contacts disposed in the connector housing, the connector contacts configured and arranged to couple to at least one of the plurality of terminals disposed on each of the at least one of the three proximal lead elements.

9. A kit for providing electrical stimulation of patient tissue, the kit comprising:
the lead of claim 1; and
a lead introducer for facilitating insertion of the lead into the patient, the lead introducer comprising
an outer member configured and arranged for insertion into the patient, and
an insertion needle disposed in the outer member, at least a portion of the insertion needle configured and arranged to receive the distal lead element of the lead.

10. The kit of claim 9, wherein at least one of the proximal lead elements defines a stylet lumen that extends along at least a portion of the longitudinal axis of the proximal lead element and that couples with the stylet lumen of the distal lead element.

11. The kit of claim 10, further comprising a stylet for facilitating guidance of the electrodes to a target stimulation region within the patient, the stylet configured and arranged for insertion into the stylet lumen of the distal lead element via the stylet lumen defined in at least one of the proximal lead elements.

12. The kit of claim 9, wherein the outer member is configured and arranged to divide into at least two parts for removal of the outer member from the lead upon insertion of the lead into the patient.

13. The kit of claim 9, wherein the insertion needle defines an open channel configured and arranged to receive at least a portion of the distal lead element of the lead.

14. The kit of claim 9, wherein the insertion needle comprises a plurality of body lead members configured and arranged to at least partially separate from one another upon removal of the outer member.

15. A method for implanting a trial electrical stimulation lead into a patient, the method comprising:
providing an insertion needle disposed in an outer member;
guiding a distal end of the outer member with the insertion needle to a target stimulation region within the patient;
inserting the distal lead element of the lead of claim 1 into at least a portion of the insertion needle;
separating the insertion needle from the lead;
removing the insertion needle from the lead; and
removing the outer member from the patient while leaving the lead within the patient such that the plurality of electrodes are at the target stimulation region;

16. The method of claim 15, wherein removing the outer member from the patient comprises separating the outer member into at least two parts along a length of a lumen of the outer member.

17. The method of claim 15, wherein removing the insertion needle from lead comprises passing the lead laterally through an open channel of the insertion needle.

18. The method of claim 15, wherein removing the insertion needle from the lead comprises at least partially separating at least two of a plurality of body lead members from one another along a longitudinal axis of the insertion needle.

19. The method of claim 15, wherein removing the outer member from the patient comprises rolling up or sliding the outer member proximally along a longitudinal axis of the insertion needle.

20. The method of claim 15, wherein removing the outer member from the patient comprises tearing up the retaining member into a plurality of pieces or splitting the retaining member along a longitudinal axis of the retaining member.
